(19) United States
(12) Patent Application Publication
(10) Pub. No.: US 2015/0031617 A1
(43) Pub. Date: Jan. 29, 2015

(54) USE OF THYMOSIN ALPHA FOR TREATMENT OF PURULENT RHINOSINUSITIS

(71) Applicant: SCICLONE PHARMACEUTICALS, INC., Foster City, CA (US)

(72) Inventor: Hemmo A. Drexhage, Rotterdam (NL)

(21) Appl. No.: 14/383,638

(22) PCT Filed: Mar. 8, 2013
(86) PCT No.: PCT/US2013/029929

§ 371 Date: Sep. 8, 2014

Related U.S. Application Data
(60) Provisional application No. 61/608,427, filed on Mar. 8, 2012.

(51) Int. Cl. A61K 38/22; A61K 47/48

(52) U.S. Cl. CPC A61K 38/2292; A61K 47/48215
USPC 514/12.9

(57) ABSTRACT

The present invention provides methods for preventing or treating purulent rhinosinusitis. These methods include administering a composition comprising thymosin alpha 1 to a subject.

### CROSS REFERENCE TO RELATED APPLICATIONS

The present application claims priority and benefit of U.S. provisional application No. 61/608,427, filed Mar. 8, 2012, which is incorporated herein by reference in its entirety.

### FIELD OF THE INVENTION

The present invention relates to the use of thymosin peptides for the treatment of purulent rhinosinusitis.

### BACKGROUND

Sinusitis includes bacterial and/or viral infection of the sinuses. Acute sinusitis typically resolves within 4 weeks with treatment. Recurrent acute (subacute) sinusitis is diagnosed after 2-4 episodes of infection occur per year with at least 8 weeks between episodes. Recurrent acute sinusitis episodes last approximately 4-8 weeks. Sinus mucosa is typically normal between infections for both acute and recurrent. Chronic sinusitis is diagnosed when there are persistent symptoms beyond 8 weeks.

Purulent rhinosinusitis can be acute or chronic and is characterized by discharge that is typically green in color and may contain pus and/or blood.

Sinusitis is often accompanied by a localized headache or toothache. Infection of the eye socket is possible, which may result in the loss of sight and can be accompanied by fever and severe illness. Another possible complication is the infection of the bones (osteomyelitis) of the forehead and as well as infection of other facial bones (Pott's puffy tumor).

Rhinosinusitis can also lead to a variety of symptoms, including facial pain, facial pressure, nasal blockage, discharge, fever, headaches, halitosis, fatigue, cough, ear pain/pressure/fullness, malaise and/or sore throat. Sinus infections can also cause additional inner ear problems due to the congestion of the nasal passages and can lead to dizziness, “a pressurized or heavy head”, or vibrating sensations in the head.

The close proximity of the brain to the sinuses makes one of the most dangerous complications of sinusitis infection of the brain by the invasion of anaerobic bacteria through the bones or blood vessels. Abscesses, meningitis and other life-threatening conditions can result from such infections. In extreme cases mild personality changes, headache, altered consciousness, visual problems, and, finally, seizures, coma, and possibly death can occur.

Sinusitis is often caused by viruses and can sometimes resolve without antibiotics. However, if symptoms do not resolve within 10 days or so, antibiotics can be used. However, these antibiotics alone are often ineffective and in some cases may be no more effective than placebos. Some studies have found 60% to 90% of people do not experience resolution of symptoms using antibiotics (see, e.g., Ian G. Williamson et al. JAMA 298 (21): 2487-96 (2007) and van Buchem, F. L.; Knottnerus, J. A., Schrijnemaekers, V. J. J., Peeters, M. F. Lancet 349 (9053): 683-7 (1997)).

Currently, for chronic or recurring sinusitis, referral to an otolaryngologist specialist may be needed, and treatment options can include nasal surgery. While short-course (3-7 days) of antibiotics can sometimes be effective for patients who present without severe disease or any complicating factors, there remains a need for additional treatments for individuals with chronic or recurring sinusitis, and in particular chronic or recurring rhinosinusitis.

The present invention provides non-invasive methods for treating and/or preventing purulent rhinosinusitis and in particular, chronic purulent rhinosinusitis.

### SUMMARY OF THE INVENTION

The present invention provides methods for prevention, amelioration, and/or treatment of purulent rhinosinusitis. Such methods for prevention amelioration and/or treatment of purulent rhinosinusitis in a subject comprise administering to the subject an effective amount of thymosin peptide, such as thymosin alpha 1 (TA1), in a composition that does not contain humoral factor. In some embodiments the composition administered is not thymostimulin (TP-1). The TA1 may be synthetic, or may be recombinantly produced.

The purulent rhinosinusitis prevented or treated by the present methods can include chronic rhinosinusitis or relapsing purulent rhinosinusitis, which may be of bacterial, viral, mixed, or unknown etiology.

In some embodiments, the subjects are tested for monocyte or granulocyte polarization, and where the subject exhibits deficient or abnormal monocyte or granulocyte polarization, the subject is administered the thymosin peptide regimen as described herein.

Therefore, in another aspect, the present invention provides for prevention and/or treatment of a monocyte or granulocyte polarization defect in a subject, comprising administering to the subject the composition or regimen of thymosin peptide as described herein, so as to induce effective or normal polarization.

The present invention also provides for a kit for treating subjects according to the thymosin peptide regimen described herein, as well as for identifying subjects in need of such treatment. In some embodiments, the kit comprises 1) a set of synthetic or recombinant thymosin peptide dosage units and 2) an instruction for administering the composition comprising thymosin peptide for treatment, amelioration, or prevention of purulent rhinosinusitis or a monocyte or granulocyte polarization defect, and/or 3) one or more reagents for testing monocyte or granulocyte polarization.

### DESCRIPTION OF THE FIGURES

FIG. 1 shows monocyte and granulocyte polarization in patients with purulent rhinosinusitis with and without treatment with thymosin alpha.

FIG. 2 shows N-formylmethionyl-leucyl-phenylalanine (fMLP)-induced polarization of healthy donor monocytes.

### DETAILED DESCRIPTION

The present invention is based in part on the surprising discovery that administering an effective amount of thymosin peptides, such as thymosin alpha 1, provides benefits for treating, preventing or ameliorating purulent rhinosinusitis. As such the present invention provides methods for preventing, treating or ameliorating purulent rhinosinusitis in a subject. These methods include administering to the subject a composition comprising an effective amount of a thymosin peptide. The composition includes a thymosin peptide in an effective concentration so as to treat or prevent purulent rhinosinusitis but does not contain humoral factor.

The subject may have purulent rhinosinusitis, or may be determined to have purulent rhinosinusitis based on the presence of one or more symptoms. Purulent rhinosinusitis is often characterized as an inflammation in one or more of the paranasal sinuses that is chronic or relapsing. The infection can result from a viral infection, bacterial infection and/or fungal infection. The infection can be of mixed or unknown etiology. Symptoms can include headaches, toothaches, infection of the eye socket, fever and in some cases infection of the bones (osteomyelitis) of the forehead and/or infection of other facial bones, in some cases referred to as Pott's puffy tumor. Additional symptoms can include inner ear problems due to the congestion of the nasal passages which can be accompanied by dizziness, pressurized or heavy head feeling and/or vibrating sensations in the head.

The purulent rhinosinusitis can be acute, chronic or relapsing purulent rhinosinusitis. Prior to treatment, the acute rhinosinusitis can be persistent for one day, two days, three days, four days, five days, 1 week, 2 weeks, 3 weeks, or 4 weeks or more. The chronic sinusitis can be persistent for about 8 weeks, about 10 weeks, about 12 weeks, about 1 month, about 2 months, about 6 months or about 1 year or more prior to treatment as described herein. Relapsing, also often referred to as recurrent, can include 4, 5, 6, 7, 8 or more recurrences of acute disease within a 12-month period. In some cases, symptoms resolve between each episode.

In some embodiments, the subject is tested for abnormal granulocyte or monocyte polarization, to determine whether the patient is an optimal candidate for thymosin peptide therapy. For example, peripheral blood monocytes may be prepared or isolated from patient blood, and polarization tested in response to an appropriate reagent, such as N-formyl-methionyl-leucyl-phenylalanine (FMLP or fMLP). Monocytes may be prepared or isolated by density gradient centrifugation, and polarization detected or quantified by light microscopy, or other appropriate technique. Where the sample exhibits FMLP-induced polarization of less than about 20%, the subject is identified as having a polarization defect or insufficient polarization, and is an optimal subject for thymosin peptide treatment. In some embodiments, the sample exhibits FMLP-induced polarization of less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2% or less than about 1%.

According to the present invention, the thymosin peptide is administered at an effective amount. In some embodiments, the thymosin peptide is thymosin alpha. An effective amount includes any amount of thymosin peptide sufficient to provide the benefits described herein. The benefits provided by administering thymosin peptide to subjects with purulent rhinosinusitis can include treatment, prevention and/or amelioration of the purulent rhinosinusitis condition and/or amelioration of symptoms associated with purulent rhinosinusitis, for example but not limited to those listed herein. Alternatively, the beneficial effects can be observed by testing patients for granulocyte or monocyte polarization. One of skill in the medical field could readily determine such beneficial effects in response to thymosin peptide treatment.

Thymosin peptides include thymosin alpha 1 (“TA1”), and peptides having structural homology to TA1. TA1 is a peptide having the amino acid sequence (N-acetyl)-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH (SEQ ID NO: 1). The amino acid sequence of TA1 is disclosed in U.S. Pat. No. 4,079,137, the disclosure of which is hereby incorporated by reference. TA1 is a non-glycosylated 28-amino acid peptide having an acetylated N-terminus, and a molecular weight of about 3108. A synthetic version of TA1 is commercially available in certain countries under the trade name ZADAXIN. The thymosin alpha 1 of the present invention also includes SEQ ID NO:1 as well as derivatives and variants thereof that exhibit similar activity to the thymosin alpha 1 of SEQ ID NO:1. The thymosin peptide composition contemplated by the methods of the present invention does not contain humoral factor. In some embodiments of the present invention the composition is not thymostimulin (TP-1).

TA1 circulates in serum at about 0.1 to 1.0 ng/ml. Peak plasma levels after injection of 3.2 mg of TA1 (about 40 μg/kg) approximately 100 ng/ml. The half-life of TA1 in the circulation is about 2 hours.

The thymosin peptides that find use with the invention include naturally occurring TA1 (e.g., TA1 purified or isolated from tissues), as well as synthetic TA1 and recombinant TA1. In some embodiments, the thymosin peptide comprises the amino acid sequence of SEQ ID NO:1 (where an acylated, e.g., acetylated, N-terminus is optional). In some embodiments, the thymosin peptide comprises an amino acid sequence that is substantially similar to TA1, and maintains the immunomodulatory activity of TA1. The substantially similar sequence may have, for example, from about 1 to about 10 amino acid deletions, insertions, and/or substitutions (collectively) with respect to TA1. For example, the thymosin peptide may have from about 1 to about 5 (e.g., 1, 2, or 3) amino acid insertions, deletions, and/or substitutions (collectively) with respect to TA1.

Thus, the thymosin peptide may comprise an abbreviated TA1 sequence, for example, having deletions of from 1 to about 10 amino acids, or from about 1 to about 5 amino acids, or 1, 2 or 3 amino acids with respect to TA1. Such deletions may be at the N- or C-terminus, and/or internal, so long as the activity of the peptide is substantially maintained. Alternatively, or in addition, the substantially similar sequence may have from about 1 to about 5 amino acid insertions (e.g., 1, 2, or 3 amino acid insertions) with respect to TA1, where the activity of TA1 is substantially maintained. Alternatively, or in addition, the substantially similar sequence may have from 1 to about 10 amino acid substitutions, where the activity is substantially maintained. For example, the substantially similar sequence may have from 1 to about 5, or 1, 2, or 3 amino acid substitutions, which may include conservative and non-conservative substitutions. In some embodiments, the substitutions are conservative. Generally, conservative substitutions include substitutions of a chemically similar amino acid (e.g., polar, non-polar, or charged). Substituted amino acids may be selected from the standard 20 amino acids or may be a non-standard amino acid (e.g., a conserved non-standard amino acid).

In some embodiments, the thymosin peptide comprises an amino acid sequence having at least 70% sequence identity to SEQ ID NO:1, while maintaining the activity of TA1. For example, the thymosin peptide may comprise an amino acid sequence having at least 80%, 85%, 90%, 91%, 92%, 93%, 94%, 95%, 96%, 97%, 98%, or 99% sequence identity to SEQ ID NO:1. The thymosin peptide may comprise an amino acid sequence having 100% sequence identity to SEQ ID NO:1. In all cases, the N-terminus may be optionally acylated (e.g., acetylated) or alkylated, for example, with a C1-C10 or C1-C7 acyl or alkyl group.

In certain embodiments, the substantially similar and homologous peptides described above may function at a level of at least about 50%, 60%, 70%, 80%, 85%, 90%, 95% or about 100% relative to TA1 (SEQ ID NO:1).

The thymosin peptides may be prepared synthetically, for example, by solid phase synthesis, or may be made recombinantly and purified by known techniques.

The thymosin peptides can also be conjugated to a water soluble polymer. Such water soluble polymers function to increase the plasma half-life of the thymosin peptide in a subject. The water soluble polymer can include polyalkylene oxide homopolymers, polyoxyethylenated polyols, copolymers of these polymers as well as block copolymers of these polymers.

In certain embodiments, the thymosin peptide is pegylated to increase its half-life in circulation. Such strategies for increasing the half-life of therapeutic proteins are well known.

The effective amount of thymosin peptides can include a dosage range from about 1 mg up to about 5 mg, or about 2 mg to about 4 mg, or about 1.6 mg to about 3.2 mg. In some embodiments, the thymosin alpha is administered from about 1 mg up to about 5 mg. The thymosin peptide may generally be administered within the range corresponding to about 0.1 to 20 mg of TA1, or about 1 to 10 mg of TA1, or about 2 to 10 mg of TA1, or about 2 to 8 mg of TA1, or about 2 to 7 mg of TA1. In some embodiments, the thymosin alpha is administered at 1, 2, 3, 4, 5, 6, 7, 8, 9, 10, 15 or 20 mg. In some embodiments, the thymosin peptide is administered in a dosage of about 1.6 mg. In some embodiments the thymosin peptide is administered in a dosage of about 3.2 mg. In some embodiments, the thymosin peptide is thymosin alpha 1. In some embodiments, the thymosin alpha 1 is administered at about 1.6 mg. In some embodiments, the thymosin alpha 1 is administered at about 3.2 mg. In some embodiments, the TA1 dose is adjusted to the size of the patient, and may be provided at from 10 to 100 μg/kg (e.g., about 20, 40, 60, or 80 μg/kg). Doses may be adjusted for the species of the subject or patient, but in each case, approximately correspond to the human equivalent of TA1 (mg/kg).

The thymosin peptide may be provided in lyophilized form, and reconstituted with sterile (e.g., aqueous) diluent prior to administration. The thymosin peptide (e.g., thymosin alpha 1, also referred to as TA1) may be administered by any effective route, including by subcutaneous injection, intramuscular injection, intraperitoneal injection, intradermal injection, intravenous injection or infusion, sublingually or orally. The thymosin peptide may be provided in lyophilized form, and reconstituted with sterile (e.g., aqueous) diluent prior to administration. In certain embodiments, the thymosin peptide is administered by subcutaneous injection or by intravenous infusion. In some embodiments the method of injection is intramuscular injection. Generally, the scheduled dose of thymosin may be administered as a single dose (e.g., injection), or may be spaced out over the course of 24 hours or less, for example, by continuous infusion or repeated injection of subdose, or the like. The scheduled dose of thymosin peptide may be administered as a single injection.

In some embodiments, the TA1 may be administered by continuous infusion. Continuous infusion of TA1 is described in detail in US 2005/0049191, the entire disclosure of which is hereby incorporated by reference. Briefly, continuous infusion of thymosin peptide maintains an effective amount of a thymosin peptide in a patient's circulatory system for a longer period. The plasma half-life of subcutaneously injected TA1 is about two hours, and thus, according to certain embodiments, the thymosin peptide may be administered to the patient for treatment periods of at least about 6, 10, 12 hours, or longer, which may improve effectiveness in some embodiments. The infusion may be carried out by any suitable means, such as by minipump.

Alternatively, the thymosin peptides can be administered by a plurality of injections (sub-doses of thymosin peptide) on a treatment day, so as to maintain an effective amount of the thymosin peptide in the patient's circulatory system for a longer period of time. Suitable injection regimens may include an injection every 2, 3, 4, 6, 8, etc. hours on the day of administration (e.g., from 2 to 5 injections), so as to substantially continuously maintain the effective amount of the thymosin peptide in the patient's circulatory system on the day of thymosin treatment.

The effective amounts of a thymosin peptide (e.g. TA1) may be substantially continuously maintained in a patient's circulatory system by administering the thymosin peptide to the patient at a rate within a range of about 0.0001-0.1 mg/hr/kg patient body weight. Exemplary administration rates are within a range of about 0.0003-0.03 mg/hr/kg patient body weight. For continuous infusion, the TA1 peptide is present in a pharmaceutically acceptable liquid carrier, such as water for injection, or saline in physiological concentrations.

In some embodiments, the treatment regimen lasts 8 weeks. In some embodiments, the first two weeks of treatment involved daily injections of thymosin. In some embodiments, injections are give twice weekly. In some embodiments, beginning with week 3, injections are given twice weekly. In some embodiments, injections are given daily for two weeks and then twice weekly beginning at week 3. In some embodiments, injections are given daily for two weeks, and twice weekly for 3 to 5 weeks. In some embodiments the treatment regimen lasts for 1, 2, 3, 4, 5, 6, 7, 8, 9, 10 or more weeks. In some embodiments, the treatment regimen lasts for 1, 2, 3, 4, 5, 6 or more months. In some embodiments, the thymosin peptide is administered in a regimen that involves daily injection of thymosin peptide for about 1 to about 4 weeks (e.g., about 2 weeks), followed by two injections per week for about 4 to 8 weeks (e.g., about 6 weeks).

The invention is applicable to both human and veterinary health. Thus, the subject is generally a mammal, such as a human, livestock (e.g., cow, horse, pig, sheep, etc.), or domestic mammal (e.g., cat or dog).

In some embodiments, the subject has asthma or allergic disease. In some embodiments, the subject is immunodeficient. An immunodeficient subject (e.g., a human subject) exhibits a reduced capacity to fight infectious disease and/or a reduced capacity to respond to pathogen exposure. Examples of such immunodeficient subjects include an elderly patient, newborn, leukemic or neutropenic patient, a patient on hemodialysis (e.g., for treatment of chronic renal disease), patient receiving immunosuppressant therapy, AIDS patient, diabetic patient, patient receiving chemotherapy or radiation therapy for cancer, immunodeficiency caused by a genetic defect, malnutrition, drug abuse, alcoholism, or other immune-compromising illness or condition.

The regimen may be administered after unsuccessful antibiotic therapy or surgery for the purulent rhinosinusitis. Where the infection is deemed resistant to antibiotics, the thymosin peptide may be administered alone, without antibiotic therapy. Alternatively, antibiotic therapy may be co-administered with the thymosin peptide regimen.

According to the present invention, thymosin peptides can be administered alone or in conjunction with other agents, such as antibiotics, antifungals or antivirals. In some embodiments, the thymosin peptide can be formulated to contain the thymosin peptide and one or more antibiotics, antifungals, antivirals, decongestants/expectorants, steroids or other agents as a single composition for administration. In other embodiments, the pharmaceutical compositions can be formulated to contain the thymosin peptide as one composition and one or more antibiotics, antifungals, antivirals, decongestants/expectorants, steroids or other agents as a separate composition for administration. In some embodiments, combinations of more antibiotics, antifungals, antivirals, decongestants/expectorants, steroids and/or other agents can be formulated with the thymosin peptide for a single administration or as a separate composition for administration.

Antibiotics can include for example but are not limited to penicillins (such as but not limited to amoxicillin or amoxicillin/clavulanate), fluoroquinolones (such as but not limited to moxifloxacin), macrolide antibiotics (such as but not limited to clarithromycin and azithromycin), a tetracycline (such as but not limited to doxycycline), or an aminoglycoside antibiotic (such as but no limited to gentamicin).

Antifungals can include for example but are not limited to amphotericin B, itraconazole and voriconazole.

Antivirals can include for example but are not limited to ribavirin.

Decongestants/expectorants can include but are not limited to guaifenesin, pseudoephedrine, or combinations such as Mucinex D (guaifenesin and pseudoephedrine HCL).

Steroids can include but are not limited to methylprednisolone, corticosteroids (such as but not limited to triamcinolone), prednisone and glucocorticosteroids (such as but not limited to mometasone furoate and dexamethasone).

Other agents can include but are not limited to dornase alfa (Pulmozyme; recombinant human deoxyribonuclease I (rhDNase) that reduces viscosity in the lungs), omalizumab (Xolair; recombinant DNA-derived humanized IgG1k monoclonal), theophylline (also known as dimethylxanthine), anesthetics (such as but not limited to propofol and sevoflurane) and opioid analgesics (such as but not limited to remifentanyl).

In some embodiments, thymosin peptides can be used in combination with sinonasal irrigation and/or surgical procedures. Sinonasal irrigation can include procedures such as irrigation with saline solutions. Surgical procedures can include removal of nasal polyps that can be caused due to prolonged or chronic inflammation.

According to the present invention, methods of prevention, amelioration or treatment of a monocyte or granulocyte polarization defect in a subject are also provided, as well as methods for inducing monocyte or granulocyte polarization. Monocyte polarization defects may be associated with a variety of diseases and conditions, including purulent rhinosinusitis as well as autoimmune diseases and conditions, including Graves' disease and thyroid autoimmune disease (TAID). These methods include administering to the subject a composition comprising thymosin alpha 1, in order to prevent, ameliorate or treat a monocyte or granulocyte polarization defect (e.g., by inducing polarization). According to these methods, the composition for administration and the administration regimen is as described above. In some embodiments, compositions comprising thymosin alpha can be used to increase monocyte polarization to normal levels, as compared to a standard level associated with an individual not affected by a polarization defect. Subjects having a defect in polarization may be identified by FMLP-induced polarization in peripheral blood monocytes as described herein.

The present invention also provides for a kit comprising 1) a set of thymosin peptide in a dosage unit and 2) an instruction for administering the composition comprising thymosin peptide for treatment, amelioration, or prevention of purulent rhinosinusitis, or a monocyte or granulocyte polarization defect, and/or 3) one or more reagents for testing polarization. In some embodiments, the thymosin peptide in the composition is thymosin alpha 1, with a sufficient number of doses provided for administering the treatment regimen described herein. The doses may be provided in conventional vials, or provided as individual dosage units (e.g., pre-dosed pens for subcutaneous injection). The reagents for testing polarization may include one or more of FMLP and sample tubes sufficient for density gradient centrifugation. In some embodiments, the thymosin peptide in the kit does not contain humoral factor. In some embodiments, the thymosin peptide in composition is thymosin alpha 1 and the composition does not contain humoral factor.

### EXAMPLES

### Example 1

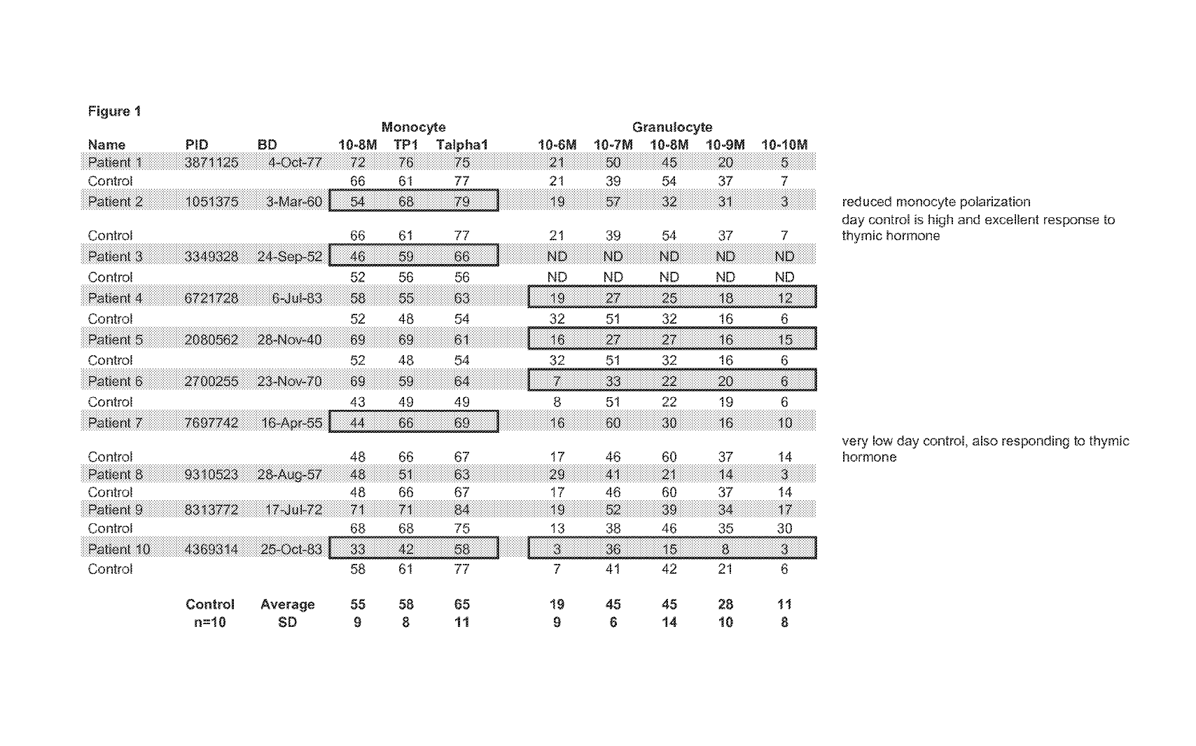

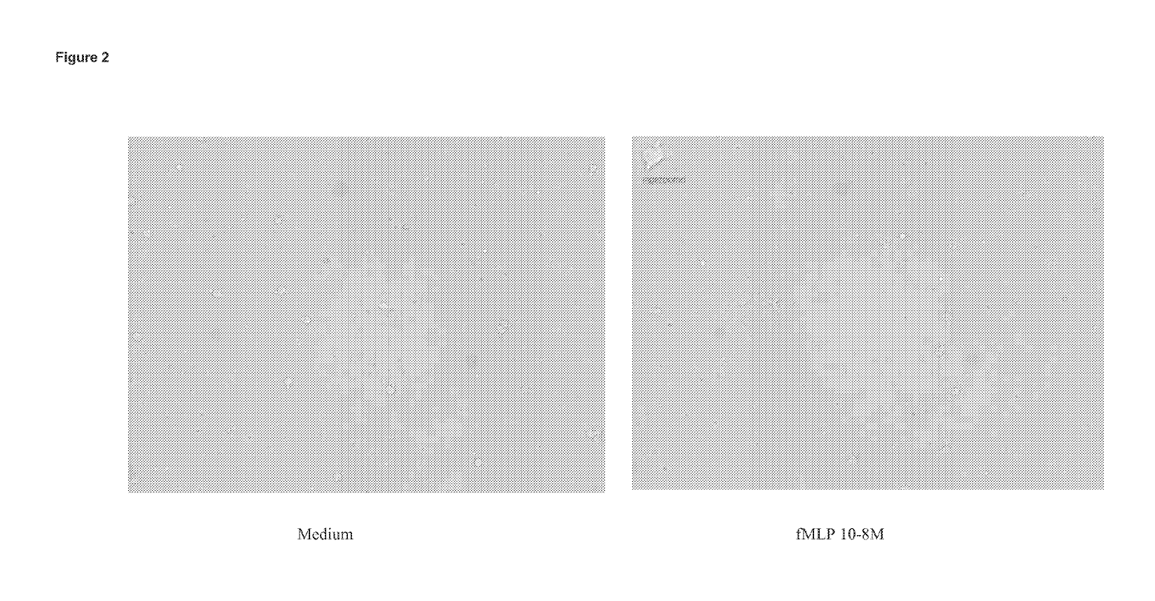

The results in 10 patients were evaluated. The monocyte and granulocyte polarizations in 10 patients were evaluated. Initially, 3 to 4 of the patients started with decreased polarization. Thymostimulin (TP-1) showed improvement in polarization in vitro. In particular, thymosin alpha (thymalfasin) worked excellently in improving and restoring the polarization in vitro. (See, FIG. 1.) As a control, N-formylmethionyl-leucyl-phenylalanine (fMLP)-induced polarization of healthy donor monocytes. (See, FIG. 2.) Concentrations were TP-1, 1 mg/ml, thymalfasin, 0.1 mg/ml and fMLP, 10⁻⁸M.

In vivo, patients with reduced monocyte polarization with thymosin alpha 1 will be treated in a pilot study to determine the effects on monocyte polarization in vivo.

### Example 2

Patients with purulent rhinosinusitis are treated with thymosin alpha 1. Similar to the study described in Tas, et al. (Clin. Exp. Immunol. 80:304-313 (1990)), patients for inclusion in this study will be screened using the following criteria (see, e.g., Drexhage et al., Clin. Immunol. Immunopathol. 28:218 (1983) and Recent Advances in Primary and Acquired Immunodeficiencies, vol. 28 (ed. by F. Aiuti, F. Rosen & M. D. Cooper) p. 395. Serono Symposia Publications, Raven Press, New York (1985)):
(1) a defective cell-mediated immunity (CMI) towards commensal microorganisms as indicated by (a) a defective monocyte polarization at previous testing, and/or (b) a defective skin test towards candidin, Streptokinase-Streptodornase (Sk/Sd) and/or H. influenzae antigen at previous testing, and/or (c) a defective MIF-production towards candidin, Sk/Sd and/or H. influenzae at previous testing;
(2) relapsing of chronic purulent rhinosinusitis as indicated by (a) duration of the disease of at least 18 months, (b) a positive culture for H. influenzae, S. pneumoniae, other Streptococci, or Staphylococci at one or more occasions, (c) no response to or only temporary relief after treatment with several courses of antibiotics, (d) failure of surgery to give a permanent cure by improving the drainage of the ethmoidal and maxillary sinus, (e) no gross disturbances in mucociliary transport.

Patients will have normal levels of total serum IgG, IgM and IgA, normal total numbers of peripheral blood leucocytes and a normal differential lymphocyte count.

Additional treatment with antibiotics and/or other drugs known to influence the immune system will not be given during the thymosin alpha trial. However, subsequent combination therapies may be applied.

Patients will give informed consent and the ethical committee of the hospital where the trial will be undertaken will approve the trial.

The treatment will consist of an intramuscular injection of either thymosin alpha 1 at (1 mg/kg body weight) or its placebo [solvent (pyrogen-free sodium chloride solution)+carrier (mannitol); specifically prepared by Serono]. Patients will be randomly allocated to two groups; one group starting with thymosin alpha 1 injections, the other with placebo injections.

Treatment will be given for 8 weeks. For the first 2 weeks injections will be given daily, and that will be followed by twice a week for 6 weeks. On week 9 no treatment will be given in order to test patients' cell-mediated immunity.

Thereafter the schedule will be “crossed over”: thymosin alpha 1 will be given to patients who had previously been given a placebo, and placebo will be given to patients who had previously been given thymosin alpha 1. Treatment will be given for 8 more weeks.

The week prior to the trial the following tests will be carried out for each patient (for techniques see below): (1) an inspection of the nose, (2) a bacterial culture from the nose, (3) a BSR, (4) DTH skin tests with bacterial antigens, (5) lymphocyte subset determinations in the peripheral blood, (6) an MIF assay, (7) a polarization assay with peripheral blood monocytes, (8) a determination of the P15E-like factor in serum.

Tests 1, 2, 3, 7 and 8 will be repeated at week 4, 8, 13 and 17 (at the end of the trial). Tests 4, 5 and 6 will only be repeated at week 8 (before cross-over) and at week 17 (end of trial).

In all patients, parameters relevant to liver function (bilirubin, ALAT, ASAT) and kidney functions (creatinine, proteinurea) will be checked during treatment.

DTH Skin Tests.

The following skin test antigens will be used (see also Drexhage et al., Recent Advances in Primary and Aquired Immunodeficiencies, vol. 28 (ed. by F. Aiuti, F. Rosen & M. D. Cooper) p. 395. Serono Symposia Publications, Raven Press, New York (1985)): (1) 250 pg/ml of a somatic H. influenzae antigen, prepared as described (Drexhage et al., 1983; van der Plassche-Boers et al., J. Immunol. Methods, 83:353 (1985)); (2) two commercially available preparations; viz., 1% Candidal antigen (HAL allergens, Haarlem, The Netherlands); and 100 U/ml Sk and 50 U/ml Sd (Varidase, Lederle, Wayne, Mich.).

Delayed responsiveness will be tested by intradermal injection of 0.1 ml suspension of each antigen preparation in the forearm. The skin reactions will be read at 30 minutes, 6 hours, 24 hours and 48 hours and the diameter of the induration, expressed as the average of two measurements at right angles, will be recorded.

Enumeration of Total Peripheral Blood Lymphocytes and Lymphocyte Subsets.

The percentage of lymphocytes and lymphocyte subsets will be determined by reacting peripheral blood lymphocytes isolated by Ficoll-Isopaque density gradient centrifugation (Pharmacia, Uppsala, Sweden) with CD3+ antibodies against T cells (Leu 4; Becton Dickinson, Mountain View, Calif.), CD2+ antibodies against active T cells (OKT I 1; Orthoclone Ortho, Raritan, N.J.), CD4+ helper/inducer T cells (Leu 3a), CD8+ suppressor/cytotoxic T cells (Leu 2a), and CD24+ B cells (BA-1; Hybritech, San Diego, Calif.) as indicated by the manufacturer. Approximately two-hundred cells will be counted in a fluorescent microscope; the tests will be done in duplicate. Absolute numbers of T cells and T cell subsets will be calculated by multiplication from the total peripheral lymphocyte counts.

Macrophage Migration Inhibition Factor Test.

Macrophage inhibitory factor (MIF) production will be estimated with an indirect microdroplet agarose assay (for details, see, van der Plassche-Boers et al. Clin. Exp. Immunol. 66:516 (1986)). Briefly, peripheral blood mononuclear cells (approximately 2.5×10⁶) will be cultured with the antigens of H. influenzae, Candidin and Sk/Sd. Supernatants will be prepared using the mitogen Concanavalin A (Con A, Sigma, St Louis, Mo.). Supernatants will also be collected after 3 days of culture (37° C., 5% CO₂ in air) and can be stored at −20° C. until testing for MIF activity.

The agarose microdroplet assay will be performed according to Thurman et al. (1983) using the human monocytoid U937 as indicator cells (Singh & Khan, J. Clin. Hemat. Oncol. 12:29 (1982)). From the cells (approximately 2×10⁷ cells/ml) in 0.2% agarose (Marine Colloids, Rockland, USA) 1 μL droplets will be centrally placed in the wells of flat-bottomed microtitre plates (Nunc, Denmark) using a Hamilton Repeating Dispenser with a 0.05 mL gas-tight syringe (Hamilton, Reno, Ark.). The droplets will be left to solidify at 4° C. for 10-20 minutes, and will be carefully overlaid with 0.1 ml of thawed supernatant diluted 1:1 with fresh medium. Each supernatant will be tested five times.

After incubation of the covered plates for 21 hours at 37° C., and 5% CO₂ in air, migration areas (cell migration area minus area of the agarose droplet) will be computed. MIF production will be expressed as percent migration inhibition:

MI=100−((Mean migration area in antigen-stimulated cultures)/(Mean migration area in medium))×100%

The Isolation of Peripheral Blood Monocytes and the Polarization Assay.

Peripheral blood mononuclear cells (20×10⁶) isolated by Ficoll-Isopaque density gradient centrifugation will be washed twice in phosphate-buffered saline (PBS), pH 7.4 containing 0.5% bovine serum albumin (BSA), and then will be counted in suspension employing positive staining with non-specific esterase (Mullink et al., J. Immunol. Methods, 29:133 (1979)). The percentage of NSE-positive cells varied, at 5-25%. An enrichment for the monocytes in the Ficoll-Paqueisolated fraction will be obtained by Percoll gradient centrifugation (Pertoft et al., J. Immunol. Methods 33:22 (1980)). After washing, the pellet containing the monocytes will be resuspended in the above-mentioned medium and carefully underlayed with an equal volume of Percoll 1.063 (Pharmacia, Uppsala, Sweden). After centrifugation (40 min, 450 g) the cells will be collected from the interface, washed twice in medium (10 minutes, 500×g) and counted: the suspension now contained 70-95% NSE-positive cells.

The Cianciolo & Snyderman assay for monocyte polarization will be performed with slight modifications (see, e.g., Tan et al., Arch. Otolaryngol. Head Neck Surg. 112:541 (1986)); 0.2-ml aliquots of the Percoll- or electicator-purified cell suspension containing 0.2×10⁶ monocytes will be added to 12-75 mm polypropylene tubes (Falcon Labware Division of Becton Dickinson Co., Oxnard, Calif.) containing 0.05 mL of either medium or N-formyl-methionyl-leucyl-phenylalanine (FMLP) in medium, to reach a final concentration of 10 nM. All experiments will be carried out in duplicate. The tubes will be incubated at 37° C. in a waterbath for 15 minutes. The incubation will be stopped by addition of 0.25 ml ice-cold 10% formaldehyde in 0.05% PBS (pH 7.2). The cell suspensions will be kept at 4° C. until counting in a haemocytometer using an ordinary light microscope (magnification 250×). The test will be read ‘blindly’ by two persons and 200 cells will be counted from each tube. A cell will be considered ‘polarized’ if any of the following occur: (1) elongated or triangular shape, (2) broadened lamellopodia, (3) membrane ruffling.

The percentage of polarized monocytes will be calculated as follows:

((% total cells polarized×100%)/(% NSE-positive cells))×100%

Lymphocytes will not exhibit any polarization activity in this assay (Cianiolo & Snyderman, J. Clin. Invest. 67:60-68 (1981)).

The chemotactic responsiveness of a monocyte population is expressed as the percentage of polarized monocytes in the presence of FMLP minus the percentage of polarized monocytes in the absence of FMLP. The assay has proven to be a rapid method to test monocyte chemotaxis and outcomes of the assay correlate well with outcomes of the conventional Boyden chamber assay to measure chemotaxis (Tan et al., Arch. Otolaryngol. Head Neck Surg. 112:541 (1986)). FMLP-induced polarization values of less than 20% will be considered to be abnormal.

Determination in Patient Serum of Low Molecular Weight Factors (LMWFs) Inhibiting Monocyte Polarization.

Sera will be collected from the patients by venepuncture and diluted 1:1 in saline. These dilutions will be subjected to ultrafiltration for 15 minutes at 700 g (molecular weight ‘cut off point’ will be 25 kD). The residues will be resuspended and stored at −70° C. until further use.

The capability of the serum fractions to inhibit FMLP induced polarization of healthy donor monocytes will be determined by incubating the monocytes (1×10⁶/ml) during 15 minutes at 37° C. either with FMLP alone or with FMLP in combination with a serum fraction (final dilution 1:60).

Addition of serum fractions alone to donor monocytes will not affect the polarization. The percentage of inhibition of FMLP-induced minus spontaneous polarization caused by addition of the serum fractions will be calculated.

The in vitro effects of anti-PiSE monoclonal antibodies and thymosin alpha 1 on the activity of serum LMWFs. In this series of experiments, elutriator-purified monocytes (de Boer & Roos, J. Immunol. 136:3447 (1986)) of one healthy donor will be used as an indicator system. In brief, mononuclear cells will be separated from 450 ml whole blood via percoll centrifugation (20 min, 1000 g, room temperature). Thereafter the mononuclear cells will be injected into an elutriation centrifugation system (Beckman J21 centrifuge with a JE-6 elutriation rotor). The elutriation medium will be PBS with 13 mM trisodium citrate and 5 mg of human albumin per ml. To separate the different cell populations, the flow rate will be kept constant at 20 ml/min while the rotor speed will be diminished from 4000 to 0 rev/min. The fraction at 2500 rev/min was collected. After Percoll gradient centrifugation this fraction contains 93-97% monocytes as judged by positivity for non-specific esterase activity. Monocytes will be stored in liquid nitrogen until use.

With this indicator system the P15E-like character of the LMWFs will be validated. Adsorption experiments will be carried out by incubating the serum fractions with a combination of two P15E-specific monoclonal antibodies (see below) in a final dilution of 1:200 at 4° C. for 16 h, followed by Amicon ultrafiltration to remove formed complexes; this adsorption procedure will be carried out twice (Tan et al., Arch. Otolaryngol. Head Neck Surg. 112:541 (1986) and Tan et al., Arch. Otolaryngol. Head Neck Surg. 112:942 (1986)). The monoclonal antibodies used will be a combination of 4F5 and 19F8 (anti-PI SE isotypes IgG2a and IgG2b; kindly provided by Dr G. J. Cianciolo, Genentech Inc., Pharmacological Sciences, South San Francisco, Calif.). As a control antibody anti-human IgG will be used (Tago, Burlingame, Calif.).

Patient serum fractions of <25 kDa diluted in culture fluid or incubated with thymosin (final dilution 67 jug/ml) and serum fractions after adsorption with the monoclonal antibodies will be tested in the monocyte polarization assay. Thymosin alpha 1 will lead to an increase in monocyte polarization.

All publications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the appended claims.

SEQUENCES

SEQUENCE: 1
Ser Asp Ala Ala Val Asp Thr Ser Ser Glu Ile Thr
1 5 10
Thr Lys Asp Leu Lys Glu Lys Lys Glu Val Val Glu
15 20
Glu Ala Glu Asn
25

1. A method for prevention, amelioration or treatment of purulent rhinosinusitis in a subject, comprising administering to the subject a composition comprising an effective amount of thymosin peptide, so as to treat, ameliorate or prevent purulent rhinosinusitis, wherein the composition does not contain humoral factor.

2. The method of claim 1, wherein the thymosin peptide is thymosin alpha 1 (TA1).

3. The method of claim 1, wherein the composition is not thymostimulin (TP-1).

4. The method of claim 2, wherein the TA1 is synthetic or recombinant.

5. The method of claim 1, where the subject has chronic or relapsing purulent rhinosinusitis.

6. The method of claim 1, wherein the purulent rhinosinusitis is of bacterial, viral, mixed, or unknown etiology.

7. The method of claim 1, wherein the subject has tested positive for abnormal monocyte polarization prior to administration of thymosin peptide.

8. The method of claim 1, wherein administration of the thymosin peptide results in increased monocyte polarization.

9. The method of claim 1, wherein the subject has been treated unsuccessfully with antibiotic and/or surgery for the purulent rhinosinusitis.

10. The method of claim 1, wherein the thymosin peptide is administered with antibiotics and/or after surgery for the purulent rhinosinusitis.

11. The method of claim 1, wherein the subject is not administered antibiotics.

12. The method of claim 1, wherein the thymosin peptide is administered at a dose of from about 1 mg up to about 5 mg.

13. The method of claim 12, wherein the thymosin peptide is administered at a dose of about 1.6 mg.

14. The method of claim 12, wherein the thymosin peptide is administered at a dose of about 3.2 mg.

15. The method of claim 1, wherein the thymosin peptide is administered by injection or infusion.

16. The method of claim 1, wherein the thymosin peptide is administered by intramuscular or subcutaneous injection.

17. The method of claim 1, wherein the thymosin peptide is conjugated to a water soluble polymer to increase the plasma half-life of the thymosin peptide in said subject.

18. The method of claim 1, wherein the thymosin peptide is administered by daily injection for about 2 weeks followed by two injections per week for about 6 weeks.

19. A method for prevention, amelioration or treatment of a monocyte or granulocyte polarization defect in a subject, comprising administering to the subject a composition comprising an effective amount of thymosin peptide, so as to induce a monocyte or granulocyte polarization, wherein the composition does not contain humoral factor.

20. A method for increasing monocyte or granulocyte polarization in a subject with a monocyte or granulocyte polarization defect, comprising administering to the subject a composition comprising an effective amount of a thymosin peptide, so as to increase monocyte or granulocyte polarization, —wherein the composition does not contain humoral factor.

21. The method of claim 19, wherein the subject tests positive for a monocyte or granulocyte polarization defect prior to administration of the thymosin peptide.

22. A kit comprising 1) a set of synthetic or recombinant thymosin peptide dosage units and 2) an instruction for administering the composition comprising thymosin peptide for treatment, amelioration, or prevention of purulent rhinosinusitis or a monocyte or granulocyte polarization defect, and/or 3) one or more reagents for testing monocyte or granulocyte polarization.

23. The method of claim 20, wherein the subject tests positive for a monocyte or granulocyte polarization defect prior to administration of the thymosin peptide.


SEQUENCE LISTING (1 sequence)

SEQ ID NO: 1 - protein, 28 aa, Artificial Sequence
  THYMOSIN ALPHA POLYPEPTIDE
SDAAVDTSSEITTKDLKEKKEVVEEAEN